(19) United States
(12) Patent Application Publication
(10) Pub. No.: US 2007/0000070 A1
(43) Pub. Date: Jan. 4, 2007

(54) Method and kit for applying lowlights to hair

(76) Inventors: Lou Ann Christine Vena, Scotch Plains, NJ (US); Teresita Vergara Imperial, Staten Island, NY (US); Dalal Ibrahim Esber Duffer, North Brunswick, NJ (US); Saroja Narasimhan, Matawan, NJ (US)

Correspondence Address: Julie Blackburn;Revlon Consumer Products Corporation, 237 Park Avenue, New York, NY, 10017, US

(21) Appl. No.: 11/172,537

(22) Filed: Jun. 30, 2005

(51) Int. Cl. A61K 8/00

(52) U.S. Cl. 8/405

(57) ABSTRACT

A kit and method for applying lowlights to hair

### TECHNICAL FIELD

The invention is in the field of applying color to hair and kits used for applying color to hair.

### BACKGROUND OF THE INVENTION

Many women are interested in not just coloring their hair, but applying gradations of color to hair. Hair highlighting is a very common procedure where portions of the hair are treated with a composition that causes the treated portions of the hair to be lighter in color than the base hair shade. Highlighting can be done in a professional salon or at home using retail hair highlighting kits. While in the past, the best quality highlights were obtained in a professional salon environment, now, the kits that are available for purchase at retail by consumers are well designed, enabling the at home user to obtain salon-equivalent results.

In recent years lowlighting has become popular. Lowlighting is a form of hair coloring where portions of the hair are treated with a composition that causes the treated portions of hair to be darker, or have a different tonality and hue of color than the base hair shade. An example of lowlighting hair would involve applying red or burgundy streaks to dark brown hair, or honey colored streaks to light blonde hair. Consumers often like to highlight their hair in the warm weather months and lowlight the hair in the fall and winter season. While retail highlighting kits are widely available in the mass market channel for at home use, there are no such kits available for lowlighting in that channel of trade. Accordingly, lowlighting procedures must be typically performed in a professional salon environment. Given the expense of salon lowlighting procedures, consumers will often forego the lowlighting process because they do not wish to spend that amount of money, and the more economical consumer friendly retail kits for at-home users are not currently commercially available.

While it would be possible for a consumer who desired to apply lowlights to hair to purchase a standard hair color kit in the desired shade and apply the hair color mixture to only select strands of hair, such hair color kits are not well designed for this purpose. First, standard hair color kits do not contain an applicator suitable for use in applying the mixture to select hair strands. Second, the viscosity of a lowlighting composition must be within very specific ranges to ensure that the composition can be applied to hair in the desired patterns and remain where applied without running or bleeding. If the lowlighting composition applied to select strands of hair bleeds onto other hair strands, the end result will not provide the gradations of color desired. Third, as lowlighting compositions are often a similar color to the base hair color shade, it would be very difficult for the consumer to see where she applied the composition. As the desired lowlighting effect is to provide gradations in hair color, if the lowlight mixture is not applied on select strands of hair the effect does not look natural or professional.

Accordingly, it is desirable to provide retail kits specifically designed for lowlighting hair which address such consumer issues.

It is an object of the invention to provide a kit for lowlighting hair comprising: (a) a first container containing an aqueous based oxidizing agent composition; (b) a second container containing a lowlight dye composition; (c) an applicator for applying the mixture of (a) and (b) to select strands of hair to provide gradations of deeper color approximating lowlights to the hair; wherein the mixture applied to hair contains a particulate in an amount sufficient to provide a visually distinctive appearance when applied to the hair.

It is a further object of the invention to provide a kit for lowlighting hair comprising: (a) a first container containing an aqueous based semi-permanent lowlighting dye composition containing at least one particulate in an amount sufficient to provide a visually distinctive appearance when applied to hair; and (b) an applicator for applying (a) to hair to provide gradations of deeper color approximating lowlights to the hair.

It is a further object of the invention to provide a method for lowlighting hair by combining an aqueous oxidizing agent composition and an oxidative dye composition and applying this mixture to select strands of hair to cause gradations of color in the hair that approximate lowlights; wherein the mixture applied to hair contains one or more particulates in an amount sufficient so that the mixture applied to hair is visually distinct from the base hair color, thus enabling the consumer to see where the lowlight mixture is being applied to the hair.

It is a further objective of the invention to provide a method for lowlighting hair by applying to select strands of hair an aqueous based semi-permanent dye composition containing at least one particulate in an amount sufficient to provide a visually distinctive appearance when applied to the hair.

### SUMMARY OF THE INVENTION

The invention comprises a kit for lowlighting hair comprising: (a) a first container containing an aqueous based oxidizing agent composition; (b) a second container containing a lowlight dye composition; (c) an applicator for applying the mixture of (a) and (b) to select strands of hair to provide gradations of deeper color approximating lowlights to the hair; wherein the mixture applied to the hair contains at least one particulate in an amount sufficient to provide a mixture that provides a visually distinctive appearance when applied to the hair.

It is a further object of the invention to provide a kit for lowlighting hair comprising: (a) a first container containing an aqueous based semi-permanent lowlighting dye composition containing at least one particulate in an amount sufficient to provide a visually distinctive appearance when applied to hair; and (b) an applicator for applying (a) to hair to provide gradations of deeper color approximating lowlights to the hair.

A method for applying lowlights to hair comprising the steps of:

(a) combining at least an aqueous oxidizing agent composition and a lowlight dye composition,

(b) applying the mixture of (a) to select strands of hair using an applicator,

(c) leaving the mixture on the hair for a period of time ranging from 1 to 60 minutes,

(d) rinsing the mixture from the hair;

wherein the mixture of (a) provides a visually distinctive appearance when applied to the hair enabling the user to determine where the mixture was applied to the hair.

The invention is also directed to a method for lowlighting hair by applying to select strands of hair an aqueous based semi-permanent dye composition containing at least one particulate in an amount sufficient to provide a visually distinctive appearance when applied to the hair.

The invention is further directed to a method for applying lowlights to hair comprising treating the hair with a lowlight dye mixture containing particulates in an amount sufficient to provide a composition having a visually distinctive appearance when applied to the hair.

The invention is further directed to a lowlight dye composition containing at least one particulate in an amount sufficient to give the composition a coloration that is visually distinctive from the color of natural hair.

### BRIEF DESCRIPTION OF THE DRAWINGS

FIG. 1: depicts the various components that may be found in one type of lowlighting kit of the invention where the lowlights are obtained from oxidative dyes.

FIG. 2: depicts another type of lowlighting kit of the invention where the lowlights may be applied with a semi-permanent dye composition, and the types of components that may be found in the kit.

FIG. 3: shows how the lowlighting mixture is applied to the hair by first loading the mixture onto the applicator for application to the hair.

FIG. 4: depicts one embodiment of the method of the invention showing how the applicator is used to apply the lowlighting mixture to the hair, and the visually distinctive appearance of the streaks applied to hair, enabling the user to see where the mixture was applied. In this embodiment, holding the applicator in a sideways manner is preferred for application of the lowlighting mixture to the hair.

FIG. 5: depicts other types of applicators that may be found in the lowlighting kit or the combination highlighting and lowlighting kit of the invention.

### DETAILED DESCRIPTION

When used herein the term “lowlight” means that the streaks of color applied to the hair are darker or provide a different tonality or hue than that of the hair color shade. Preferably, that when the lowlight dye composition is applied to the hair it provides streaks of color that are at least one shade darker than that of the base hair shade. In the chart set forth below the different levels are identified and examples of the primary intermediates and color couplers that are known to be useful in providing the particular level of color are set forth. It is noted that nothing herein shall be construed to limit the scope of the invention to the particulate primary intermediates and couplers set forth, but rather they are set forth for purposes of exemplification only:

| Level 1 - Very Black | | Level 2 - Bright Black | |
|---|---|---|---|
| Primary Intermediates | Couplers | Primary Intermediates | Couplers |
| p-phenylenediamine | m-aminophenol | p-phenylenediamine | resorcinol |
| p-phenylenediamine | resorcinol | 2-chloro-P-phenylenediamine | |
| sulfate | | sulfate | |
| 2-chloro-phenylenediamine | 4-amino-2-hydroxytoluene | o-aminophenol | |
| sulfate | | | |
| p-aminophenol | 4-chlororesorcinol | | |
| o-aminophenol | m-aminophenol HCL | | |
| | 2,4-diaminophenoxyethanol | | |
| | m-phenylenediamine | | |
| | sulfate | | |

| Level 3 - Very Dark Brown | | Level 4 - Dark Brown | |
|---|---|---|---|
| Primary | | Primary | |
| Intermediates | Couplers | Intermediates | Couplers |
| p-phenylenediamine | resorcinol | p-phenylenediamine | resorcinol |
| N,N-bis(2- | 1-naphthol | N,N-bis(2- | 1-naphthol |
| hydroxyethyl)-P- | | hydroxyethyl)-P- | |
| phenylenediamine | | phenylene diamine | |
| sulfate | | sulfate | |
| | m- | p-aminophenol | m-aminophenol |
| | aminophenol | | |
| | | | phenyl methyl |
| | | | pyrazolone |
| | | o-aminophenol | 4-amino-2- |
| | | | hydroxytoluene |

| Level 5 - Medium Brown | | Level 6 - Light Brown | |
|---|---|---|---|
| Primary Intermediates | Couplers | Primary Intermediates | Couplers |
| p-phenylenediamine | resorcinol | p-phenylenediamine | resorcinol |
| N,N-bis(2-hydroxyethyl)- | 1-naphthol | N,N-bis(2-hydroxyethyl)- | 1-naphthol |
| P-phenylenediamine | | P-phenylenediamine | |
| sulfate | | sulfate | |
| p-aminophenol | m-aminophenol | p-aminophenol | m-aminophenol |
| o-aminophenol | phenyl methyl | | phenyl methyl |
| | pyrazolone | | pyrazolone |
| | 2-methylresorcinol | | 4-amino-2-hydroxytoluene |
| | 4-amino-2-hydroxtoluene | | 2-methylresorcinol |

| Level 7 - Dark Blonde | | Level 8 - Medium Blonde | |
|---|---|---|---|
| Primary | | Primary | |
| Intermediates | Couplers | Intermediates | Couplers |
| p-phenylenediamine | resorcinol | p-phenylenediamine | resorcinol |
| N,N-bis(2- | 1-naphthol | N,N-bis(2- | 1-naphthol |
| hydroxyethyl)-P- | | hydroxyethyl)-P- | |
| phenylenediamine | | phenylenediamine | |
| sulfate | | sulfate | |
| p-aminophenol | phenyll | p-aminophenol | m-aminophenol |
| | methy | | |
| | pyrazolone | | |
| o-aminophenol | | | phenyl methyl |
| | | | pyrazolone |
| | | | 4-amino-2- |
| | | | hydroxytoluene |

| Level 9 - Light Blonde | | Level 10 - High Lift Blonde | |
|---|---|---|---|
| Primary Intermediates | Couplers | Primary Intermediates | Couplers |
| p-phenylenediamine | resorcinol | p-phenylenediamine | resorcinol |
| N,N-bis(2- | 4-amino-2-hydroxytoluene | N,N-bis(2-hydroxyethyl)- | 1-naphthol |
| hydroxyethyl)-P- | | P-phenylenediamine | |
| phenylenediamine | | sulfate | |
| sulfate | | | |
| p-aminophenol | phenyl methyl | | phenyl methyl |
| | pyrazolone | | pyrazolone |
| o-aminophenol | 2-methylresorcinol | | 2-methylresorcinol |
| | 1-naphthol | | |

By way of example, if the consumer starts with a base hair color shade of Level 10, then in one of the preferred embodiments of the invention, the method and kit will provide streaks of color that are at least a Level 9 or darker. Similarly, if the consumer starts with a base hair color shade of Level 6, medium brown, then the lowlight color applied to the hair in accordance with the most preferred method and kit of the invention would be Level 5 color or darker. Generally, if the consumer's hair color is less than Level 3, dark brown, any color applied to the hair may not provide a visually detectable end result.

The term “visually distinctive” means, with respect to the lowlight mixture (as defined below) contains one or more particulates that cause the mixture to have a color that exhibits a sufficient contrast with the consumer's base hair color such that the consumer can readily see the strands of hair to which the mixture is applied. For example, the lowlight mixture applied to hair may be bright blue, pink, red, lime green, and so on.

The term “lowlighting mixture” means the composition applied to the hair that is operative to color hair. For example, in the case where the lowlights are applied with an oxidative dye composition obtained by combining a developer and an oxidative dye composition, the lowlighting mixture is the mixture of those two compositions. In another example, if the lowlights are applied using an aqueous based semi-permanent dye composition (that does not require activation to color the hair), then the lowlighting mixture refers to the semi-permanent dye composition. In yet another example, in the case where the lowlights are applied by a hair color mixture obtained by combining a developer, oxidative dye, and particulate persulfate composition, this combination of ingredients will be the lowlighting mixture.

The components of the kit are further described herein.

I. The Oxidative Lowlighting Kit

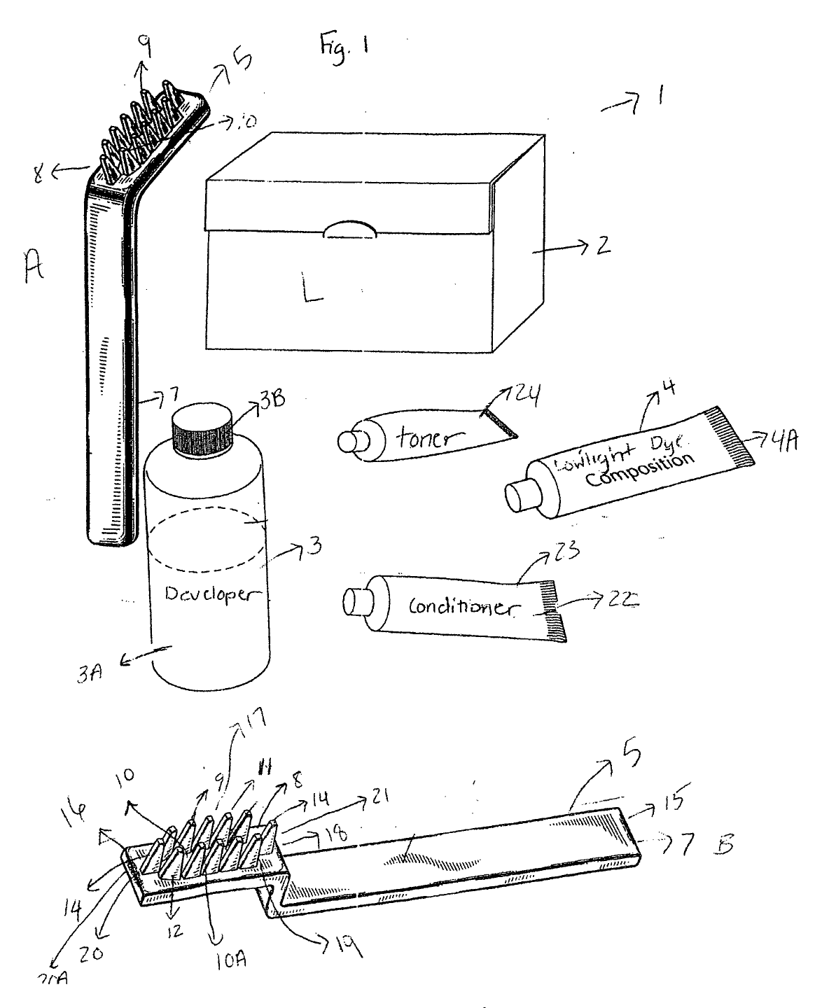

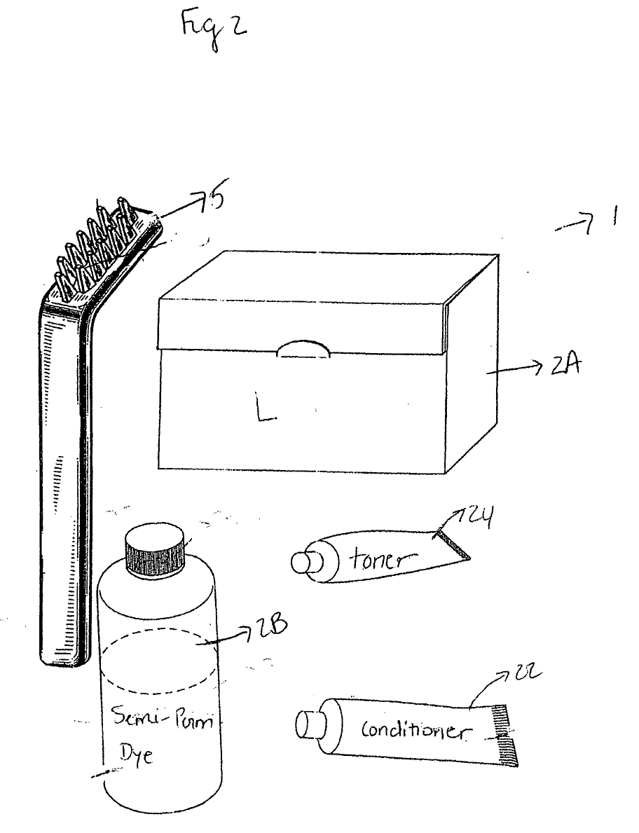

FIG. 1 depicts a lowlighting kit 1 of the invention based upon oxidative hair color, and containing the various types of components that may be found therein. FIG. 2 depicts a lowlighting kit 1 of the invention based upon semi-permanent hair color, and shows the various types of components that may be found therein.

A. Container

The lowlighting kit 1 generally comprises at least one container 2, which is preferably cardboard, plastic, paper, or similar. The container 2 may be printed with graphics or indicia, such as instructions for how to use the product, model pictures, a listing of ingredients found in the various compositions, and so on. The “L” designation on the front of the container 2 in FIG. 1 means that the kit 1 is for lowlighting hair. The container 2 must be sufficiently durable to house the other components of the kit 1. It is also important that the kit be inexpensive and easy to manufacture and print.

B. The Aqueous Oxidizing Agent Composition

The lowlighting kit 1 contains at least an aqueous oxidizing agent composition 3 (also referred to as a developer). The developer 3 is contained, preferably, in a plastic bottle 3A having a removable cap 3B, or similar type of receptacle. The term “developer” and “aqueous oxidizing agent composition” will be used interchangeably herein, and mean the same thing. The developer composition may be in the solution or emulsion form. If the latter, the emulsion may be in the water-in-oil or oil-in-water form. Further, the emulsion may also be in the microemulsion or liquid crystalline form, if desired.

When the aqueous oxidizing agent is in the solution form the composition preferably comprises about 1-40% by weight of the total composition of an oxidizing agent, preferably hydrogen peroxide, and preferably about 50-99% by weight of the total composition of water. Other water soluble ingredients may be included in the solution, such as humectants, preservatives, water soluble thickeners, antioxidants, and so on.

When the aqueous oxidizing agent composition is in the emulsion form, the composition preferably comprises about 1-40% of oxidizing agent, preferably hydrogen peroxide, about 50-99% water, and about and 0.01-30%, preferably about 0.05-20%, more preferably about 0.1-15% of an oily phase. The aqueous oxidizing agent composition may be in the form of a water-in-oil or oil-in-water emulsion or in the form of a transparent microemulsion wherein the dispersed particles in the continuous phase are so small (generally about 5-1500 Å) that the composition is optically clear. Examples of suitable microemulsion compositions are set forth in U.S. Pat. No. 6,315,989, which is hereby incorporated by reference in its entirety. It is also suitable that the aqueous oxidizing agent composition be in the form of a composition containing liquid crystals as set forth in U.S. Pat. No. 6,238,653, which is hereby incorporated by reference in its entirety.

The various ingredients that may be found in the aqueous oxidizing agent, or developer, composition further may include, but are not limited to:
1. Oxidizing Agent

Preferably the oxidizing agent is hydrogen peroxide, although other suitable peroxides such as urea peroxide, sodium perborate, etc. may be used as well. Preferably the aqueous oxidizing agent composition contains hydrogen peroxide. The oxidizing agent contributes to formation of active oxygen when the various components are combined.
2. Lipophilic Ingredients

One or more lipophilic ingredients, or oils, may be present in the aqueous oxidizing agent composition if this composition is in the emulsion form. If so, suggested ranges are about 0.1-85%, preferably about 1-70%, preferably about 2-65% by weight of the total composition. Suitable lipophilic ingredients may be liquids, semi-solids, or solids oils at room temperature (25° C.). Examples of such lipophilic materials include short chain hydrocarbons, polar hydrophilic oils, fatty acids, fatty alcohols, silicone waxes, and so on. If such other lipophilic ingredients are present, suggested ranges are about 0.1-50%, preferably about 0.5-35%, more preferably about 1-30% by weight of the total composition.
3. Humectants

Humectants may be present in the aqueous oxidizing agent composition. If so, suggested ranges are from about 0.01-10%, more preferably about 0.05-8%, most preferably about 0.1-5% by weight of the total composition of humectant. Suitable humectants include monomeric, homopolymeric, and/or block copolymeric ethers as well as mono-, di-, or polyhydric alcohols.

Suitable ethers are formed by the polymerization of monomeric alkylene oxides, generally ethylene or propylene oxide. Such polymeric ethers have the following general formula: 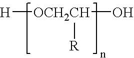
wherein R is H or lower alkyl and n is the number of repeating monomer units, and ranges from 1 to 500.

Also suitable are polyols such as glycerine or C₁₋₄ alkylene glycols and the like. Particularly preferred are C₁₋₄ alkylene glycols, in particular propylene and/or butylene glycol and ethoxydiglycol.

Suitable mono-, di-, or polyhydric alcohols include glycerin, butylene glycol, ethylene glycol, propylene glycol, and so on.
4. Water Soluble Thickeners

The aqueous oxidizing agent composition may contain one or more water soluble thickeners. If present suggested ranges are from about 0.1-25%, preferably about 0.5-20%, more preferably 1-15% by weight of the total composition. Suitable thickeners include, but are not limited to:
(a) Acrylic Copolymer Thickeners

Suitable acrylic copolymeric thickeners are comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof. Preferably, the A monomer comprises one or more of acrylic acid or methacrylic acid, and the B monomer comprises is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. Most preferably, the acrylic copolymer is supplied in an aqueous solution having a solids content ranging from about 10-60%, preferably 20-50%, more preferably 25-45% by weight of the polymer, with the remainder water. Preferably, the thickening agent is a polymer comprised of A, B, and C monomers wherein A and B are as defined above, and C has the general formula: 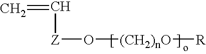
Preferably, in the copolymer used for the secondary thickening agent in the preferred embodiment of the invention, A and B are as above defined; and in the C monomer Z is (CH₂)ₘ, m is 1-2, n is 2, and o is 2-100, and R is a C₁₂₋₂₂ straight or branched chain alkyl. More preferably in the C monomer m is 1, n is 2, o is 10, and R is C₁₈ or stearyl, and the compound is steareth-10 allyl ether/acrylate copolymer, which may be purchased from Allied Colloids under the tradename Salcare SC90 or SC80.

Also suitable is an aqueous solution of an acrylic polymer comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof. Preferably, the A monomer comprises one or more of acrylic acid or methacrylic acid, and the B monomer comprises is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. Most preferably, the acrylic copolymer is supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Preferably, the acrylic copolymer contains enough of the A monomer to enable ionization in a basic solution, thereby causing the ionized carboxylic acid groups in the polymer to repel each other, and thereby “swallow” water. Particularly preferred acrylic copolymer solutions suitable for use in the developer composition include those sold by Seppic, Inc., under the tradename Capigel, in particular, Capigel 98, which is a white liquid having a pH of 2 to 4, a solids content of about 29-31, a density of 1.04 to 1.08, and a viscosity of 700-1000 millipascal seconds at 25° C.
(b) Associative Thickeners

Various other types of associative thickeners may be present, including water soluble urethane homo- and copolymers, and the like.
(c). Other Thickeners

Also suitable for use as thickening agents are carboxyvinyl polymers, also referred to as Carbomer, generally described as homopolymers of acrylic acid crosslinked with a polyfunctional agent, such as the allyl ether of sucrose, allyl ether of pentaerythritol, or allyl ether of propylene.

Also suitable are various types of cellulosic based thickeners or substituted cellulosic based thickeners, including but not limited to hydroxyethyl cellulose, hydroxypropyl cellulose, xanthan gum, and the like.
5. Nonionic Surfactants

If desired, the aqueous developer composition may contain one or more nonionic surfactants. Recommended ranges are 0.01-10%, preferably 0.05-8%, more preferably 0.1-7% by weight of the total composition.
(a) Alkoxylated Alcohols

Suitable nonionic surfactants include alkoxylated alcohols, or ethers, formed by the reaction of an alcohol with an alkylene oxide, usually ethylene or propylene oxide. Preferably the alcohol is a fatty alcohol having 6 to 30 carbon atoms, and a straight or branched, saturated or unsaturated carbon chain. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Particularly preferred is Ceteareth 20, which is the reaction product of a mixture of cetyl and stearyl alcohol with ethylene oxide, and the number of repeating ethylene oxide units in the molecule is 20.
(b) Alkoxylated Carboxylic Acids

Also suitable as the nonionic surfactant are alkyoxylated carboxylic acids, which are formed by the reaction of a carboxylic acid with an alkylene oxide or with a polymeric ether. The resulting products have the general formula: 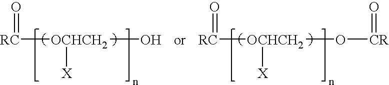
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.
(c) Sorbitan Derivatives

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

The aqueous oxidizing agent composition may also comprise a variety of other ingredients including cationic, amphoteric, or zwitterionic surfactants, preservatives.

C. Oxidative Lowlight Dye Composition

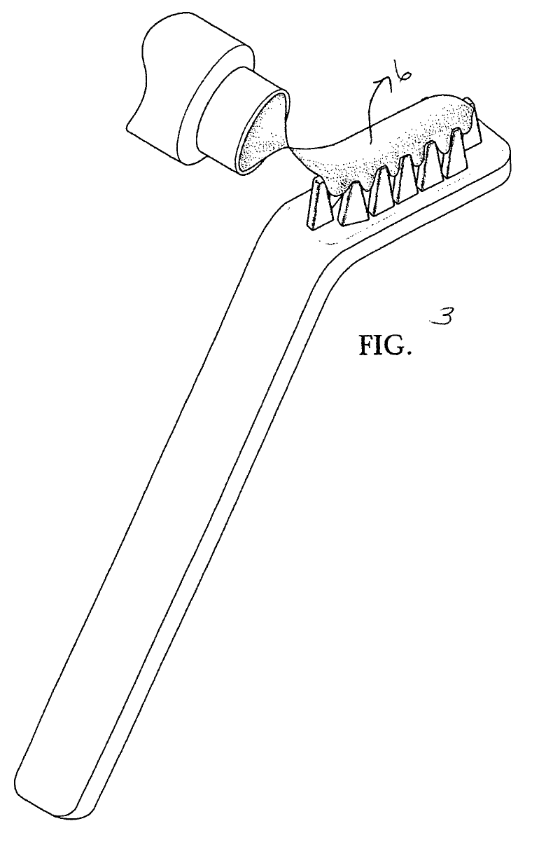

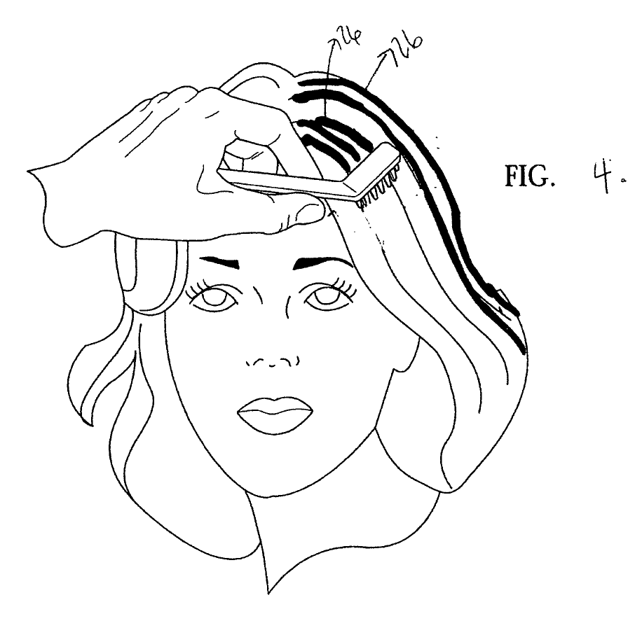

The lowlighting kit also contains a lowlight dye composition 4. The lowlight dye composition 4 may be stored in a tube 4A or any other suitable storage receptacle such as a packette or bottle. The lowlight dye composition 4 contains at least one primary intermediate and, optionally, at least one coupler for the formation of oxidation dyes, and is an aqueous based composition. Specifically, the lowlight mixture 6 is prepared by combining the developer composition 3 and the lowlight dye composition 4, preferably by adding the lowlight dye composition 4 to the container in which the developer composition 3 is found. The components are mixed well to form the lowlighting mixture 6 (see FIG. 3). The lowlight mixture 6 is loaded into the applicator 5 as depicted in FIG. 3. The applicator 5 containing the mixture 6 is then stroked through the hair as depicted in FIG. 4, to deposit the mixture 6 onto select strands of hair to provide lowlights. In particular, lowlights are streaks of more deeply colored hair that are intermixed with the consumer's base hair color shade, which is usually lighter. The result is hair that exhibits subtle gradations in color from lighter to darker. Generally the color gradations that result after the lowlighting procedure will be in the same general family so as to give the most natural appearance.

The lowlight dye composition may be in the form of a liquid or creme. The term “creme” means a viscous liquid or semi-solid that does not readily drip when applied to the hair. It may be in the solution or emulsion form. The lowlight dye composition is in the form of an emulsion, more preferably a water-in-oil or oil-in-water emulsion. In the case where the lowlight dye composition is in the form of emulsion, preferred is where it is an oil-in-water emulsion. Such types of emulsions may contain from about 35-98% water and 2-65% oil, all percentages by weight of the total composition. The lowlight mixture may also contain other ingredients, as set forth herein.
1. Primary Intermediates.

The lowlight dye composition comprises one or more primary intermediates. Suggested ranges of primary intermediates are 0.0001-6%, preferably 0.0005-5.5%, more preferably 0.001-5% by weight of the total composition. Such primary intermediates are known for use in oxidative hair color, and include ortho or para substituted aminophenols or phenylenediamines, such as para-phenylenediamines of the formula: 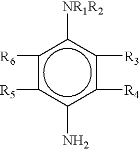
wherein R₁ and R₂ are each independently hydrogen, C₁₋₆ alkyl, or C₁₋₆ alkyl substituted with one or more hydroxy, methoxy, methylsulphonylamino, aminocarbonyl, furfuryl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₆ alkyl substituted with one or more hydroxy or amino groups.

Specific examples of suitable primary intermediates include para-phenylenediamine, 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, and derivatives thereof, and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.
2. Color Coupler

The dye mixture preferably comprises from about 0.0001-10%, more preferably 0.0005-8%, most preferably 0.001-7% by weight of the total composition of one or more color couplers which are dyestuff components. Suitable color couplers include, for example, those having the general formula: 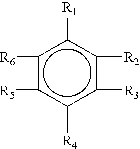
wherein R₁ is unsubstituted hydroxy or amino, or hydroxy or amino substituted with one or more C₁₋₆ hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄ together may form a methylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methyl pyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3 [(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamionphenoxyethanol, and mixtures thereof.

Preferred couplers include resorcinol, 1-naphthol, 2-methylresorcinol, 4-amino-2-hydroxy toluene, m-aminophenol, 2,4-diaminophenoxyethanol, phenyl methyl pyrazolone, their salts, or mixtures.
3. Alkalizing Agent

If desired, the lowlighting dye composition may contain one or more alkalizing agents preferably in a range of about 1-5% based on the total weight of the lowlighting dye composition. The term “alkalizing agent” means an ingredient that is capable of imparting alkalinity (e.g. a pH of greater than 7) to the lowlighting composition. Suitable alkalizing agents include ammonium hydroxide, metal hydroxides, alkanolamines, sodium silicate, metal carbonates, sodium metasilicate, and mixtures thereof. Suitable metal hydroxides and carbonates include alkali metal and alkaline earth metal hydroxides or carbonates. Examples of such metal hydroxides include sodium, potassium, lithium, calcium, magnesium and so on. A particularly preferred alkaline earth metal hydroxide is sodium hydroxide. Suitable alkanolamines include mono-, di-, and trialkanolamines such as monoethanolamine (MEA), diethanolamine (DEA), triethanolamine (TEA), 2-aminobutanol, aminoethyl propanediol, aminomethyl propanediol, bis-hydroxyethyl tromethamine, diethanolamine, diethyl ethanolamine, diisopropanolamine, dimethylamino methylpropanol, dimethyl MEA, isopropanolamine, methylethanolamine, mixed isopropanolamines, triisopropanolamine, tromethamine, and mixtures thereof. A particularly preferred alkanolamine is MEA.
4. Other Ingredients

The lowlighting dye composition may contain one or more of lipophilic ingredients such as fatty acids, hair conditioning ingredients, surfactants, and the like.
(a) Fatty Acids.

If present, the lowlighting dye composition may contain one or more fatty acids in an amount of about 0.001-15%, preferably 0.005-10%, most preferably 0.01-8% by weight of the total composition. If fatty acids are present they will react with the alkalizing agent to form soap in situ, which provides a more shampoo-like character to the lowlighting dye composition when it is combined with the developer composition and applied to hair. Such fatty acids are of the general formula RCOOH wherein R is a straight or branched chain, saturated or unsaturated C₆₋₃₀ alkyl. Examples of suitable fatty acids include oleic acid, stearic acid, myristic acid, linoleic acid, and so on. Particularly preferred is oleic acid.
(b) Conditioners

Preferably the dye mixture comprises one or more conditioners that exert a conditioning effect on hair. A variety of conditioners are suitable including cationic polymers, oily conditioning agents, fatty alcohols, proteins, and so on. A combined total weight of conditioners ranges from about 0.1-25%, preferably 0.5-20%, more preferably 1-15% by weight of the total composition. Examples of such conditioners include cationic polymers, quaternary derivatives of cellulose, copolymers of vinyl pyrrolidone, silicones such as dimethicone, cyclomethicone, phenyl trimethicones and so on. Also suitable are various types of oily conditioning agents such as esters, hydrocarbons, and the like. Preferably, the composition comprises 0.001-20%, more preferably 0.005-15%, most preferably 0.01-10% by weight of the total composition of such oils. Particularly preferred oily conditioning agents are oils extracted from vegetable sources, specifically meadow foam seed oil.
(c) Surfactants or Emulsifiers

The lowlighting dye composition of the invention preferably comprises one or more surfactants that assist in maintaining the composition in the preferred emulsion form and aid in the foaming capability of the composition. Suitable surfactants include anionic surfactants, nonionic surfactants, amphoteric surfactants, and the like. Suitable surfactants include nonionic, amphoteric, zwitterionic, or betaine surfactants. If present, suggested ranges of nonionic surfactant are about 0.01-10%, preferably about 0.05-8%, more preferably about 0.1-7% by weight of the total composition.
(i) Nonionic Surfactants

Suitable nonionic surfactants include alkoxylated alcohols or ethers, alkoxylated carboxylic acids, sorbitan derivatives, and the like.

Suitable alkoxylated alcohols, or ethers, are formed by the reaction of an alcohol with an alkylene oxide, usually ethylene or propylene oxide. Preferably the alcohol is a fatty alcohol having 6 to 30 carbon atoms, and a straight or branched, saturated or unsaturated carbon chain. Examples of such ingredients include Steareth 2-30, which is formed by the reaction of stearyl alcohol and ethylene oxide where the number of repeating ethylene oxide units is 2 to 30; Oleth 2-30 which is formed by the reaction of oleyl alcohol and ethylene oxide where the number of repeating ethylene oxide units is 2 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Particularly preferred are Steareth-21, which is the reaction product of a mixture of stearyl alcohol with ethylene oxide, and the number of repeating ethylene oxide units in the molecule is 21, and Oleth-20 which is the reaction product of oleyl alcohol and ethylene oxide wherein the number of repeating ethylene oxide units in the molecule is 20.

Also suitable as the nonionic surfactant are alkyoxylated carboxylic acids, which are formed by the reaction of a carboxylic acid with an alkylene oxide or with a polymeric ether. The resulting products have the general formula: 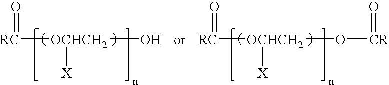
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable are various types of alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.
(ii) Anionic Surfactants

If desired, the lowlighting dye composition may contain one or more anionic surfactants. Together with the soap formed by the reaction of the fatty acid and alkanolamine or metal hydroxide, the ingredients provide the composition with the characteristics of shampoo. Preferred ranges of anionic surfactant are about 0.1-25%, preferably 0.5-20%, more preferably 1-15% by weight of the total composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:
R₁—SO₃—M
wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula: 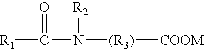
wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

Also suitable are amphoteric and zwitterionic surfactants. Examples of amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.
(d) Thickening Agents

If desired, the lowlighting dye composition contains one or more thickening agents that increase the viscosity of the composition. The amount of thickening agent, if present, may range from about 0.001-5%, preferably about 0.005-4%, more preferably about 0.005-3% by weight of the total composition.

A variety of thickening agents are suitable including low melting point waxes, carboxyvinyl polymers, and the like. Particularly preferred thickening agents are low melting point waxes such as emulsifying wax, fatty alcohols (e.g. stearyl alcohol, cetearyl alcohol, behenyl alcohol, and the like). Preferred are cetearyl alcohol and emulsifying wax.
(e) Solvents

It may be desirable to include one or more solvents in the lowlighting dye composition. Such solvents assist in solubilizing the primary intermediate, and/or coupler components, in addition to the other ingredients in the composition. The solvent is preferably present at about 0.01-10%, preferably 0.05-8%, more preferably 0.1-7% by weight of the total composition. Suitable solvents include C₂₋₄ alkanols such as ethanol, isopropanol, propanol, etc., as well as askoxydiglycols such as ethoxydiglycol. The preferred solvent comprises ethoxydiglycol.
(f) Chelating Agents

Preferably, the lowlighting dye composition contains one or more chelating agents that are capable of chelating the metal ions found in water. If water contains too many extraneous metal ions they can interfere with the coloration process. Preferred ranges of chelating agent are 0.001-5%, preferably 0.005-4%, more preferably 0.01-3% by weight of the total composition. Preferred chelating agents are EDTA, HEDTA, and sodium or potassium salts thereof.
(g) Antioxidants

The dye composition may also contain one or more antioxidants as described herein with respect to the dye composition and in the same ranges by weight.

Various other ingredients such as preservatives, botanicals, and other ingredients may also be incorporated into the lowlighting dye compositions.

D. Particulates

The lowlighting dye mixture that is applied to the hair comprises at least one particulate in an amount sufficient so that the mixture provides a visually distinctive appearance when applied to the hair. While the particulate will impart color to the lowlight dye mixture to a degree sufficient to create a visually distinctive appearance when applied to hair, the particulates used will not have any effect on the dye process or end result. The particulates will easily rinse out of the hair along with the lowlight dye mixture at the conclusion of the lowlighting procedure. The particulates are present for the purpose of coloring the dye mixture so that the consumer can see where the dye has been applied and to ensure that it is applied in the most desired lowlighting pattern. This, in turn, gives the user more control over the end result and contributes to providing salon-quality lowlights. Some suitable colors for the lowlighting dye mixture include red, pink, light green, aqua, lime green, dark blue, orange, and so on.

The particulates that provide this visually distinctive appearance to the mixture may be incorporated into any one or more of the components used to prepare the mixture, or may added separately. For example, the particulates may be found in the aqueous oxidizing agent composition, the lowlight dye composition, or in both compositions. In addition, the particulates may be found in a separate packette in the kit 1, and added to the mixture formed by combining the aqueous oxidizing agent and lowlight dye composition. Alternatively, if the kit contains other components that are combined with the aqueous oxidizing agent composition and the dye composition when those compositions are mixed, then the particulates may be found in those compositions as well. In the latter case, examples of such compositions include a separate container containing a hair conditioning composition that is combined with the aqueous oxidizing agent composition and the lowlight dye composition. The three compositions combined form the mixture, and the particulates may be found in the hair conditioning composition. Alternatively, the lowlighting kit may also contain a persulfate bleach composition if the lowlighting method is conducted as set forth in U.S. Pat. No. 6,596,035, which is hereby incorporated by reference in its entirety. In this case, the particulates that provide the visually distinctive appearance may also be found in the persulfate composition that is combined with the oxidative dye composition and the hydrogen peroxide composition when the three ingredients are combined immediately prior to application to hair.

In all cases, the particulates may be found in any one or more of those compositions and the amount of particulates found in any one or more of the compositions combined to form the lowlight dye mixture must be present in an amount sufficient to ensure that the ultimate mixture applied to the hair provides a visually distinctive appearance when applied to the hair. Generally, this amount may range from about 0.0001-30%, preferably from about 0.005-25%, more preferably from about 0.001-20% by weight of the total mixture applied to hair. This means that any one or more of the compositions that are combined to form the mixture may contain the particulates in amounts ranging from about 0.0001-50%, preferably from about 0.005-45%, more preferably from about 0.001-30% by weight of the total composition. Preferably, the particulates have particle sizes ranging from about 0.001 to 200, preferably from about 0.005 to 150, more preferably from about 0.01 to 100 microns in diameter. Suitable particulates may include organic pigments, inorganic pigments, particulate fillers, or mixtures thereof.
1. Organic Pigments

If organic pigments are used in the mixture to provide the visually distinctive appearance on hair, they are most preferably water insoluble. The pigment water insolubility is generally created by reaction with various metallic salts such as calcium, aluminum, barium, zirconium and the like, to provide what are referred to as the “Lakes”. Examples of water insoluble organic pigments include Lakes of red, green, blue, yellow, violet, orange, and mixtures thereof. Particularly suitable water insoluble organic pigments are the aluminum Lakes of the organic pigments, formed by the reaction of the organic pigment with aluminum to form the water insoluble aluminum Lake. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 C.F.R. §74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 C.F.R. §82.51. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.
2. Inorganic Pigments

The particulates used to provide the visually distinctive appearance may be inorganic pigments. Suitable inorganic pigments include iron oxides such as red, blue, black, green, and yellow; titanium dioxide, bismuth oxychloride, and the like. Preferred are iron oxides. As with the organic pigments, most preferably the inorganic pigments are water insoluble.
3. Particulate Fillers

It is possible that the particulates may include particulate fillers, provided they either provide, or contribute to providing, the visually distinctive appearance. Most preferably such particulate fillers are water insoluble.

Suitable particle fillers include titanated mica, fumed silica, spherical silica, polymethylmethacrylate, micronized teflon, boron nitride, acrylate copolymers, aluminum silicate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof.

The above mentioned fillers may be surface treated with lecithin, amino acids, mineral oil, silicone oil or various other agents either alone or in combination, which coat the powder surface and render the particles more water insoluble.

In the most preferred embodiment of the invention the particulates are iron oxides, and are found in the lowlight dye composition.

E. Applicator

The kit 1 also contains at least one applicator 5 for applying the lowlight mixture to the hair. Specifically, the lowlight mixture 6 is prepared by combining the aqueous oxidizing agent composition 3 and the lowlight dye composition 4, preferably by adding the lowlight dye composition 4 to the container in which the aqueous oxidizing agent composition 3 is found. The components are mixed well to form the lowlighting mixture 6 (see FIG. 3). The lowlight mixture 6 is loaded into the applicator 5 as depicted in FIG. 3. The applicator 5 containing the mixture 6 is then stroked through the hair as depicted in FIG. 4, to deposit the mixture 6 onto select strands of hair to provide lowlights. As can be seen in FIG. 4, the strands to which the lowlighting mixture 6 was applied are differently colored and exhibit a visually distinct appearance in the form of distinct differently colored streaks 26 on the hair strands. In particular, while the actual lowlight dye will provide streaks of more darkly colored hair intermixed with the consumer's lighter base hair color shade upon completion of the procedure, the mixture streaks 26 are preferably brightly colored and visually distinct from the base hair color.

The end result is that treated hair will exhibit subtle gradations in color from lighter to darker. Preferably the color gradations that result after the lowlighting procedure will be in the same general family so as to give the most natural appearance.

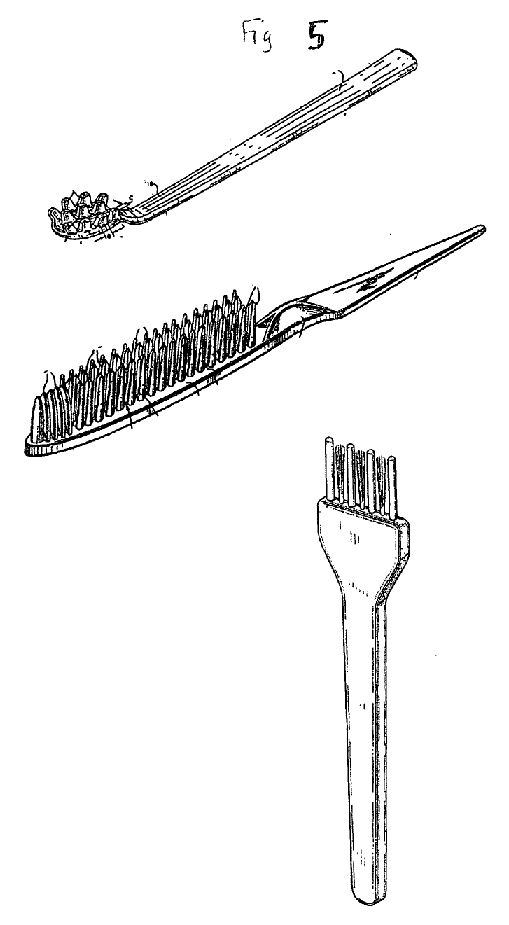

There are a variety of types of applicators that may be found in the lowlighting kit 1 of the invention. FIG. 1 depicts two types of applicators. FIG. 5 depicts additional types of applicators that may be suitable. In general the applicator 5 will have a handle 7, a head 8, and a plurality of prongs or tines 9 on the head 8 onto which the lowlight mixture is loaded prior to combing onto the select hair strands. Preferably, the prongs or tines 9 are perpendicular to the head. More preferably, the tines 9 are perpendicular to the head and together form a retaining structure 10 for the lowlighting mixture 6 such that the mixture is applied into the retaining area formed by the aligned prongs or tines.

Any applicator having this general configuration is suitable for use in applying the lowlight mixture 6 to the hair. Types of applicators that may be suitable include those set forth in U.S. Patent Publication Nos. 2005/0066989 A1, U.S. Pat. No. 6,872,228 B1, U.S. Pat. Nos. 6,453,909 B1, 6,142,157; U.S. Design 405,614; all of which are hereby incorporated by reference in their entirety, as well as those set forth in FIG. 4.

Preferred is an applicator 1 where the protrusions or prongs 9 are perpendicular to the head 8. More preferred is where the applicator depicted in FIG. 1A has a handle 7 that is sloped at an obtuse angle with respect to the head 8. More particularly, the handle 7 forms an angle ranging from about 95 to 180 degrees with respect to the head. The prongs 9 may be in a variety of shapes and sizes. For example, suitable prongs or protrusions 9 may be pillow shaped, tooth shaped, pyramid shaped, needle shaped, arrow shaped, peg shaped, and so on. Preferably, the prongs 9 are in the form of tines that form pyramid shaped tooth like protrusions 11 that are perpendicular to the head 8. The pyramid shaped teeth 11 have a wide base 12 where they are affixed to the head 8 of the applicator 1. The front and back walls 13 of the long side of the rectangular wide base 12 being generally triangular in shape and with a squared top 14. Sidewalls 14 between the front and back walls 13 are generally rectangular. The top surface of the pyramid shaped teeth preferably has a squared top 14, although such top may also be rounded or otherwise curved. Handle 7 has a proximal end 15 and a distal end 16. The distal end 16 has the head 8, which contains pyramid shaped teeth 11, which form retaining structure 10. The retaining structure 10 formed by the placement of pyramid shaped teeth 11 may be circular, oblong, rectangular, or any configuration that provides a retaining structure 10 that is adequate to hold the lowlighting mixture 6. In the most preferred embodiment of the invention the retaining structure 10 is oblong 10A (see FIG. 1), and formed by from four to six, preferably five pyramid shaped teeth 11 on each long side, with from one to two pyramid shaped teeth 11 on each end. More preferably, as depicted in FIG. 1, there are four pyramid shaped teeth 11 parallel to each other forming the long side 17 of the oblong 10A shaped retaining structure 10. Toward the bottom edge 18, facing the proximal end of applicator 1, two pyramid shaped teeth 11 are placed to slope inwardly 19 from the parallel pyramid shaped teeth 11 forming the long side 17 of the oblong 10A retaining structure 10. One pyramid shaped tooth 11 is placed at the top 20 of the retaining structure 10 and one at the bottom 21 of retaining structure 10. In the most preferred embodiment of the invention, the pyramid shaped tooth 11 at the top 20 of the retaining structure 10 is placed generally in the middle section 20A between the two parallel lines of pyramid shaped teeth 11 that form the long side 17 of the oblong 10A retaining structure 10. Additionally, in the preferred embodiment of the invention the pyramid shaped tooth 11 at the bottom 18 of the retaining structure 10 is placed generally parallel to pyramid shaped tooth 11 at the top 20, and they are generally aligned longitudinally. This configuration of the pyramid shaped teeth 11 to make retaining structure 10, which provides an area where the lowlighting mixture 6 is placed. Pyramid shaped teeth 11 may be of any length and width, and may depend on the size of the head and other factors. Preferably, pyramid shaped teeth 11 range from about 0.1 to 1.0, preferably 0.2 to 0.75 inch in length.

F. Hair Conditioner

The kit 1 may contain one or more optional components such as hair conditioner 22. The hair conditioner 22 may be found in a tube 23 or packette, or any other similar type of single or multiple use package. Generally, hair conditioner compositions are aqueous based and comprise from about 0.01-99%, preferably about 0.05-85%, more preferably about 0.1-80% by weight of the total composition of water, and from about 0.01-99%, preferably from about 0.05-85%, more preferably from about 0.1-80% of one or more hair conditioning agents which are the same as those identified above with respect the aqueous oxidizing agent composition and/or the lowlighting dye composition.

G. Toner

The kit may also contain a toner 24. In the most preferred embodiment the kit contains a toner 24, which is a composition in the shampoo form that contains at least one dye that is reactive with oxidizing agent to form color, but is free of an oxidizing agent reactive with such oxidative dyes to form color. Accordingly, when the toner is applied to the hair, the oxidative dyes present in the toner react with the residual oxidizing agent on hair to form color. Because the toner does not contain any oxidizing agents it is not operable to color hair by itself. Instead, when the toner, which is free of oxidizing agents, is applied to the entire head of hair, the dye present in the toner will react with the residual oxidizing agent that is left on the hair fibers that were treated with the lowlighting mixture. The treated hair fibers will become colored, but the untreated hairs will remain uncolored. Preferably, the toner is in the form of a shampoo so that the consumer can simply shampoo the hair for the desired period of time after the lowlighting mixture (or highlighting mixture, in the case where the kit of the invention is a combination highlighting/lowlighting kit as further described herein) has been rinsed from the hair. Most conveniently, the toner is applied to the entire head of hair, rather than just the bleached strands, yet the oxidative dyes found therein will be reactive only with the hair strands that were treated with the lowlighting mixture and have residual oxidizing agent thereon. The toner will not have any impact on the untreated hair. Examples of such toners are set forth in U.S. Patent Application Publicaton No. 2004/0154108, which is hereby incorporated by reference in its entirety.

The toner may contain a variety of ingredients in addition to the at least one oxidative dye (as described above with respect to the lowlighting composition and in the same general percentage ranges, by weight of the total toner composition. The toner is an aqueous based composition comprising, by weight of the total composition, from about 0.1-99% water, at least one primary intermediate and at least one coupler for the formation of oxdative dye, and preferably, at least one surfactant or emulsifier. Suitable primary intermediates and couplers for use in the toner are as set forth with respect to the lowlight dye composition and in the same general ranges. The surfactant, if present, generally assists in maintaining the composition in the preferred emulsion form and aid in the foaming capability of the composition. Suitable surfactants include anionic surfactants, nonionic surfactants, amphoteric surfactants, and the like, as described above with respect to the lowlighting dye composition and/or the developer composition and in the same general ranges.

Other optional components of the kit may include rubber gloves, written instructions, coupons, and a variety of other items that facilitate use of the kit by the consumer. The retail consumer purchases the kit and is able to apply salon quality lowlights to her hair using the kit components.

II. The Semi-Permanent Lowlighting Kit

A semi-permanent dye composition may also be used to apply lowlights to hair. A kit suitable for applying lowlights to hair using a semi-permanent dye composition is depicted in FIG. 2. The various components of the kit I include:

A. Container

The lowlighting kit 1 generally comprises at least one container 2A, which is preferably cardboard, plastic, paper, or similar. The container 2A may be printed with graphics or indicia, such as instructions for how to use the product, model pictures, a listing of ingredients found in the various compositions, and so on. The “L” designation on the front of the container 2A in FIG. 1 means that the kit 1 is for lowlighting hair. The container 2A must be sufficiently durable to house the other components of the kit 1. It is also important that the kit be inexpensive and easy to manufacture and print.

B. Semi-Permanent Dye Composition

The semi-permanent dye composition 2B comprises a variety of ingredients. Generally the dye composition is in an aqueous solution or emulsion form containing from about 0.01-99%, preferably from about 5-95%, more preferably from about 10-85% by weight of the total composition of water, in addition to the dyes and other ingredients mentioned.
1. Water Soluble or Dispersible Dyes

The semi-permanent dye composition contains one or more water soluble or water dispersible dyes that are operable to color hair when contact therewith. The term “operable to color the hair when contacted therewith” means, in the most preferred embodiment of the invention, that the dye alone, without combining with any additional activators or accelerators, will color the hair (as opposed to certain types of oxidative dyes which must be combined with an activator such as hydrogen peroxide in order to impart color to the hair fiber).

A variety of dyes are suitable including direct dyes, disperse dyes, acid dyes, basic, dyes, direct, dyes, and so on. Suitable amounts of dye preferably range from about 0.001-20%, preferably about 0.005-15%, more preferably about 0.010-10% by weight of the total semi-permanent dye composition. Preferred are the compounds that fall into the general category of semi-permanent dyes. Examples of such dyes are set forth below:
(a). Basic Dyes

Suitable basic dyes include blues, browns, greens, oranges, reds, and yellows. Suitable blues include Basic Blue 3, 6, 7, 9, 26, 41, 47, and 99. Suitable browns include Basic Browns 4, 16, and 17. Suitable greens include Basic Green 1 and 4. Suitable oranges include Basic Orange 1 and 2. Suitable Reds include Basic Red 1, 2, 22, 46, 76, and 118. Suitable violets include Basic Violet 1, 3, 4, 10, 11:1, 14, and 16. Suitable yellows include Basic Yellow 11, 28, and 57.

Suitable basic dyes for use in the claimed compositions are set forth in the C.T.F.A. Cosmetic Ingredient Handbook, Eighth Edition, pages 117-124, which are hereby incorporated by reference in their entirety.
(b). HC Dyes

Also suitable for use in the compositions are various HC dyes such as blue, brown, green, orange, red, violet, and yellow. Suitable blues include HC Blue 2, 4, 5, 6, 7, 8, 9, 10, 11, 12, 13, and 14. Suitable browns include HC Brown 1 and 2. Suitable greens include HC Green 1. Suitable oranges include HC Orange 1, 2, 3, and 5. Suitable reds include HC Red 1, 3, 7, 8, 9, 10, 11, 13, and 14. Suitable violets include HC Violet 1 and 2. Suitable yellows include HC Yellow 2, 4, 5, 6, 7, 8, 9, 10, 11, 13, 14, and 15. Such HC dyes are set forth on pages 615-623 of the C.T.F.A. Cosmetic Ingredient Handbook, Eighth Edition, 2000, which is hereby incorporated by reference in its entirety.
(c). Acid Dyes

Also suitable for use in the compositions are various acid dyes such as black, blue, brown, green, orange, red, violet, and yellow. Examples of Acid Black are numbers 1 and 52. Suitable blues include Acid Blue 1, 3, 9, 62, and 74, including Lakes thereof. Examples of browns and greens include Acid Brown 13 and Acid Green 1, 25, and 50, respectively. Suitable oranges include Acid Orange 3, 6, 7, and 24. Suitable reds include Acid Red 14, 18, 27, 33, 35, 51, 52, 73, 87, 92, 95, 184, and 195. Suitable violets include Acid Violet 9 and 43. Suitable yellows include Acid Yellow 1, 3, 23, and 73. In each case the dyes may be Lakes thereof. Such Acid dyes are set forth on pages 13-23 of the C.T.F.A. Cosmetic Ingredient Handbook, Eighth Edition, 2000, which is hereby incorporated by reference in its entirety.
(d). Direct and Disperse Dyes

Also suitable are various types of dyes referred to as direct dyes or disperse dyes. Suitable direct dyes include Direct Black 51, Direct Blue 86, Direct Red 23, 80, and 81; Direct Violet 48, and Direct Yellow 12. Such direct dyes are set forth on pages 469-471 of the C.T.F.A. Cosmetic Ingredient Handbook, Eighth Edition, 2000, which is incorporated by reference in its entirety.

Suitable disperse dyes include Disperse Black 9, Disperse Blue 1, 3, and 7; Disperse Brown 1, Disperse Orange 3, Disperse Red 11, 15, and 17; and Disperse Violet 1, 4, and 15. Such disperse dyes are as set forth on 491-493 of the C.T.F.A. Cosmetic Ingredient Handbook, Eighth Edition, 2000, which is hereby incorporated by reference in its entirety.

In the preferred embodiment of the invention, HC dyes and/or Disperse dyes are used.

The semi-permanent dye composition also contains particulates, as described in Section I. D. above with respect to the oxidative lowlight composition and in the same general ranges.
2. Other Ingredients

The semi-permanent dye composition may contain additional ingredients as set forth above, with respect to the oxidative lowlighting dye composition, and in the same general ranges.

C. Applicator

The kit of FIG. 2 preferably also contains an applicator 5 for the dye composition. The applicator is as described above with respect to the kit of FIG. 1 containing the oxidative lowlighting product.

D. Toner

The kit may also contain a toner composition 24 as described above with respect to the kit containing the oxidative lowlighting dye kit.

E. Hair Conditioner

The kit may also contain a hair conditioner composition 22 as described above with respect to the kit depicted in FIG. 1, for oxidative lowlighting the hair.

III. The Method

The invention comprises a method for applying lowlights to hair, preferably providing lowlights that are at least one shade darker than the consumer's base hair color shade. Referring to the chart above, for example, if the consumer's base hair color shade is light blonde, Level 9, then suitable lowlight color would be Level 8 or less.

A. Oxidative Dye Lowlights

One embodiment of the invention is directed to a method for applying lowlights to hair comprising the steps of: (a) combining at least (i) an aqueous oxidizing agent composition and (ii) a lowlight dye composition, (b) applying the mixture of (a) to select strands of hair using an applicator, (c) leaving the mixture on the hair for a period of time ranging from 1 to 60 minutes, and (d) rinsing the mixture from the hair with water; wherein the mixture provides a visually distinctive appearance when applied to the hair, enabling the user to see where the mixture was applied.

The lowlighting mixture is applied to the hair using an applicator that provides streaks of the lowlighting mixture along select hair strands. The lowlighting mixture is left on the hair for about 1 to 60 minutes, then rinsed off with water.

If desired, after the lowlighting mixture is rinsed from the hair, the hair may be further treated with a toner composition as described herein. In particular, the hair is shampooed with the toner composition (shampoo being the preferred form) and allowed to remain on the hair for a period of time ranging from about V₂ minute to 30 minutes. The oxidative dyes present in the toner composition react with any residual oxidizing agent present on the hair strands and deposit further color. The end result here is that treatment with the toner causes further deposit of color on the lowlighted hair strands, but without affecting the hair not treated with the lowlighting mixture. The hair strands treated with the lowlighting mixture will then exhibit an extra deposit of color. This, in turn, causes the treated hair strands to retain their color longer and in a more color true manner.

After the toner is rinsed from the hair with water, it may be desirable to apply hair conditioner to the hair. If so, the hair is treated with conditioner in the usual manner. The hair is then rinsed with water.

B. Semi-Permanent Lowlights

Another embodiment of the invention comprises applying lowlights to hair using the kit of FIG. 2, where the dye composition is a semi-permanent dye. In this case, it is not necessary to activate the dye prior to applying the mixture to the hair. Rather, the semi-permanent dye composition is operable to color hair by itself when applied thereto.

The semi-permanent lowlight dye mixture is applied to the hair using applicator 5 as depicted in FIG. 4. The dye mixture is visually distinct such that the consumer can easily see where the lowlight dye mixture was applied to the hair.

After application of the semi-permanent dye mixture, the mixture is left on the hair for a period of time sufficient to provide color. The mixture is then rinsed from the hair with water. If desired, the hair can be treated with a hair conditioner composition.

C. Other Methods

In another embodiment of the invention, it is possible to provide a method for applying lowlights to hair that are at least one levels darker in color than the consumer's base hair color shade, by using other hair color processes and kits. For example, the kit and process as set forth in U.S. Pat. No. 6,596,035, which is hereby incorporated by reference in its entirety. In this process, a lowlight dye composition as described herein (also referred to as an oxidative dye composition), is combined with an aqueous hydrogen peroxide based developer as further described herein, and a particulate bleach composition, also as described herein. The mixture is prepared in the amounts and ratios as taught in the '035 patent. The mixture is applied to the hair using an applicator, preferably as described herein. The resulting lights applied to hair will be at least one levels darker than the consumer's base hair color shade.

In yet another embodiment of the invention, the lowlights may be applied to hair in accordance with U.S. Patent Application Publication No. 2002/0004957, which is hereby incorporated by reference in its entirety. In this process, an aqueous oxidizing agent composition, a persulfate bleach composition, and a semi-permanent dye composition are combined and applied to streaks of hair using an applicator of the type disclosed in this application. The mixture applied to the hair strands will provide lowlights that are at least two levels darker than the consumer's base hair color shade.

In another embodiment of the invention, lowlights may be applied to the hair by combining an aqueous oxidizing agent composition as described herein, with a persulfate bleach composition as described herein, and applying this mixture to select strands of hair using an applicator. The bleach mixture is then rinsed from the hair. A toner composition, as described herein, is then applied to the hair. The dyes present in the toner composition will react with the residual oxidizing agent present on the hair strands treated with the bleach mixture to provide color on the hair. This technology is described in U.S. Patent Application Publication No. 2004/0016064, which is hereby incorporated by reference in its entirety.

The invention will be further described in connection with the following examples which are set forth for the purposes of illustration only.

### EXAMPLE 1

An aqueous oxidizing agent composition, or developer, for use in the kits of the invention was prepared as follows:

| Ingredient | wt % |
|---|---|
| Water | QS |
| EDTA | 0.02 |
| Propylene glycol | — |
| Laureth-23 | — |
| Cetearyl alcohol/sodium lauryl sulfate/sodium | — |
| cetearyl sulfate | |
| Cetearyl alcohol | 2.94 |
| Ceteareth-20 | 0.42 |
| Sodium lauryl sulfate | 0.50 |
| Mineral oil | 0.40 |
| Metaphosphoric acid | 0.004 |
| Disodium phosphate | — |
| Hydrogen peroxide (35% aqueous solution) | 12.90 |
| Steareth-10 allyl ether acrylates copolymer | 0.30 |

The compositions were prepared by combining the ingredients and mixing well. The compositions were stored in a plastic bottle.

### EXAMPLE 2

A persulfate composition for use in certain embodiments of the invention was prepared as follows:

| Ingredient | wt % |
|---|---|
| Potassium persulfate | 38.50 |
| Sodium persulfate | 14.50 |
| Sodium metasilicate | 11.00 |
| Ammonium persulfate | 9.00 |
| Sodium chloride | 7.00 |
| Sucrose | 6.00 |
| Ultramarines | 0.25 |
| Ammonium chloride | 4.00 |
| Silica | 1.00 |
| Silica silylate | 1.00 |
| Sodium lauryl sulfate | 1.00 |
| Disodium EDTA | 1.00 |
| Hydroxyethylcellulose | 3.00 |
| Xanthan gum | 2.75 |

The composition was prepared by combining the ingredients and storing in a packette.

### EXAMPLE 3

Oxidative lowlighting dye compositions suitable for use in the kit of the invention were prepared as follows:

| | wt % | | |
|---|---|---|---|
| Ingredient | Toffee | Cinnamon | Chocolate |
| Water | QS | QS | QS |
| Erythorbic acid | 0.20 | 0.20 | 0.20 |
| Sodium sulfite | 0.50 | 0.50 | 0.50 |
| Propylene glycol | 4.00 | 4.00 | 4.00 |
| Tetrasodium EDTA | 0.60 | 0.80 | 0.80 |
| Ethanolamine | 5.00 | 5.00 | 5.00 |
| Hypnea musciformis extract, | 0.80 | 0.80 | 0.80 |
| gellidiela acerosa extract, | | | |
| sargassum filipendula | | | |
| extract, sorbitol | | | |
| P-phenylenediamine | 0.40 | 0.65 | 0.70 |
| P-aminophenol | 0.15 | 0.14 | 0.14 |
| Resorcinol | 0.49 | 0.60 | 0.65 |
| 4-amino-2-hydroxytoluene | 0.075 | 0.16 | 0.18 |
| Phenyl methyl pyrazolone | 0.02 | 0.05 | 0.05 |
| M-aminophenol | — | 0.04 | 0.04 |
| Ammonium lauryl sulfate | 2.00 | 2.00 | 2.00 |
| Oleic acid | 12.50 | 12.50 | 12.50 |
| Cetearyl alcohol | 4.00 | 4.00 | 4.00 |
| Emulsifying wax | 2.00 | 2.00 | 2.00 |
| Oleth-20 | 1.00 | 1.00 | 1.00 |
| Steareth-21 | 0.70 | 0.70 | 0.70 |
| Limanthes alba (meadowfoam) seed oil | 0.75 | 0.75 | 0.75 |
| Oleyl alcohol | 0.40 | 0.40 | 0.40 |
| Polquaternium-10 | 0.20 | 0.20 | 0.20 |
| Polyquaternium-28 | 0.50 | 0.50 | 0.50 |
| Mica, titanium dioxide | 0.15 | 0.15 | 0.15 |
| Iron oxides | 0.36 | 0.36 | 0.36 |
| Hydrolyzed wheat protein | 0.50 | 0.50 | 0.50 |
| Fragrance | 0.75 | 0.75 | 0.75 |
| Ammonium hydroxide | 4.00 | 4.00 | 4.00 |

The lowlight dye compositions were prepared by combining the ingredients and mixing well. The compositions were stored in squeeze tubes.

### EXAMPLE 4

Toner compositions for use in the kits of the invention were prepared as follows:

| | Wt % | | | |
|---|---|---|---|---|
| Ingredient | Honey | Champagne | Chestnut | Currant |
| Water | QS | QS | QS | QS |
| Citric acid | 0.001 | 0.001 | 0.001 | 0.001 |
| Erythorbic acid | 0.50 | 0.50 | 0.50 | 0.50 |
| Sodium sulfite | 0.50 | 0.50 | 0.50 | 0.50 |
| Ethoxydiglycol | 2.00 | 2.00 | 2.00 | 2.00 |
| P-phenylenediamine | 0.001 | 0.06 | 0.18 | 1.20 |
| 1-naphthol | 0.003 | 0.04 | 0.12 | — |
| Resorcinol | — | 0.04 | 0.12 | — |
| 4-amino-2-hydroxytoluene | — | — | — | 0.60 |
| 2-methyl-5- | — | — | — | 0.60 |
| hydroxyethylaminophenol | | | | |
| Hydroxypropylmethyl | 0.30 | 0.30 | 0.30 | 0.30 |
| cellulose | | | | |
| Tetrasodium EDTA | 0.30 | 0.30 | 0.30 | 0.30 |
| Sodium lauryl sulfate (30% | 10.00 | 10.00 | 10.00 | 10.00 |
| aqueous solution) | | | | |
| Sodium laureth sulfate | 20.00 | 20.00 | 20.00 | 20.00 |
| (28% aqueous solution) | | | | |
| Cocamidopropyl betaine | 4.00 | 4.00 | 4.00 | 4.00 |
| Ethanolamine | 2.30 | 2.30 | 2.30 | 2.30 |
| Isostearic acid | 5.00 | 5.00 | 5.00 | 5.00 |
| Lauramide DEA | 2.00 | 2.00 | 2.00 | 2.00 |

The compositions were prepared by combining the ingredients and mixing well. They were stored in squeeze tubes.

### EXAMPLE 5

A hair conditioner compositions suitable for use in the kit of the invention were prepared as follows:

| Ingredient | Wt % |
|---|---|
| Water | QS |
| Polyquaternium-10 | 0.20 |
| Methyl paraben | 0.20 |
| Panthenol | 0.01 |
| Behentrimonium chloride | 1.50 |
| Propyl paraben | 0.05 |
| Cetearyl alcohol | 6.00 |
| Cetyl alcohol | 1.00 |
| Dicaprylyl ether | 1.00 |
| Oleyl alcohol | 1.00 |
| Mangifera indicia (Mango) seed butter | 0.001 |
| Behentrimonium methosulfate, quaternium-33, | 2.00 |
| cetyl alcohol | |
| Jojoba seed oil | 0.20 |
| Amodimethicone, trideceth-12, cetrimonium chloride | 2.00 |
| Methylchloroisothiazolinone, methylisothiazolinone | 0.04 |
| Citric acid | 0.007 |
| Sodium benzotriazolyl butylphenol sulfonate, buteth-3, | 0.01 |
| tributyl citrate | |
| Cholesteryl oleyl carbonate, cholesteryl chloride, | 0.001 |
| cholesteryl nonanoate | |
| Fragrance oil | 0.30 |
| Glycerin | 2.50 |

The composition was prepared by combining the ingredients and mixing well.

### EXAMPLE 6

Semi-permanent dye based hair color compositions suitable for applying lowlights to hair were were prepared as follows:

| | | | Dark |
|---|---|---|---|
| | Medium | Light | Brown/ |
| Ingredient (%) | Brown | Auburn | Black |
| Water | 70.85 | 73.294 | 69.145 |
| Ethoxydiglycol | 5.00 | 5.00 | 5.00 |
| Methylparaben | 0.20 | 0.20 | 0.20 |
| Disodium EDTA | 0.05 | 0.05 | 0.05 |
| Aminomethyl propanol | 3.60 | 3.60 | — |
| Monoethanolamine | — | — | 3.60 |
| Erythorbic acid | 0.06 | 0.06 | 0.06 |
| HC Yellow #2 | 0.43 | 0.126 | 0.86 |
| HC Yellow #4 | 0.43 | 0.33 | 0.805 |
| HC Red #3 | 0.15 | 0.28 | 0.10 |
| HC Blue CP | 1.97 | — | 1.97 |
| HC Red 13 | 0.30 | — | 0.25 |
| Disperse Orange #3 | — | 0.60 | — |
| HC Violet 1,3 bis | — | — | 1.00 |
| Veegum | 1.00 | 0.50 | 1.00 |
| Oleic acid | 9.38 | 9.38 | 9.38 |
| Cetearyl alcohol | 3.00 | 3.00 | 3.00 |
| Polawax | 1.50 | 1.50 | 1.50 |
| Oleth-20 | 0.75 | 0.75 | 0.75 |
| Steareth-21 | 0.53 | 0.53 | 0.53 |
| Oleyl alcohol | 0.30 | 0.30 | 0.30 |
| Fragrance | 0.50 | 0.50 | 0.50 |

The compositions were prepared by placing the water in a beaker having a homogenizer mill attached and heating to 70-75° C. The homogenizer mill was then turned on and the veegum sprinkled into the beaker. The mixture was milled for 15 minutes. While maintaining the temperature, ethoxydiglycol, disodium EDTA, erythorbic acid and methylparaben were added to mixture, which was further milled for about 15 minutes. The dyes were then added to the mixture. In a separate beaker the aminomethylpropanol, monoethanolamine, and water were combined, then this mixture was added to the mixture of other ingredients in the beaker. The composition was further milled for 30 minutes to ensure that the dyes were completely solubilized. The batch was transferred to a turbine/sweep kettle.

Separately, the oil ingredients—oleic acid, cetearyl alcohol, emulsifying wax, oleth-20, oleyl alcohol, and steareth-21—were combined and heated to 70-75° C. The oil phase was then combined with the other ingredients and mixed well while maintaining the temperature at 70-75° C. The batch was cooled to 35-40° C. The fragrance oil was added and the composition mixed for an additional 15 minutes. The batch was cooled to 25° C.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A kit for lowlighting hair comprising: (a) a first container containing an aqueous based oxidizing agent composition; (b) a second container containing a lowlight dye composition; (c) an applicator for applying the mixture of compositions in (a) and (b) to select strands of hair; wherein the mixture applied to hair contains a particulate in an amount sufficient to provide a visually distinctive appearance when the mixture is applied to the hair.

2. The kit of claim 1 wherein the container is a carton.

3. The kit of claim 2 wherein the container is a cardboard carton having graphics and indicia printed thereon.

4. The kit of claim 1 wherein the aqueous based oxidizing agent composition comprises a hydrogen peroxide developer composition.

5. The kit of claim 4 wherein the hydrogen peroxide developer composition comprises, by weight of the total composition, from about 0.01-95% water and from about 0.01-55% hydrogen peroxide.

6. The kit of claim 1 wherein the lowlight dye composition is an aqueous based colorant composition comprising at least one primary intermediate and at least one coupler for the formation of oxidative dye.

7. The kit of claim 6 wherein the lowlight dye composition comprises, by weight of the total composition, from about 0.001-25% of the at least one primary intermediate and at least one coupler for the formation of oxidative dyes, from about 0.01-99% water.

8. The kit of claim 1 wherein the applicator comprises a handle and a face containing a plurality of tines.

9. The kit of claim 1 wherein the particulate is an organic pigment, inorganic pigment, or mixture thereof.

10. The kit of claim 9 wherein the organic pigment is a water insoluble D&C or FD&C color.

11. The kit of claim 10 wherein the organic pigment is a Lake.

12. The kit of claim 9 wherein the pigment is an inorganic pigment.

13. The kit of claim 12 wherein the inorganic pigment is titanium dioxide or iron oxides.

14. The kit of claim 1 wherein the particulate is present in the oxidizing agent composition or the lowlight dye composition or both in an amount ranging from about 0.0001-15% by weight of the total composition.

15. The kit of claim 14 wherein the particulate is present in the lowlight dye composition.

16. The kit of claim 15 wherein the particulate comprises iron oxides or ultramarine blue.

17. A kit for lowlighting hair comprising: (a) a first container containing an aqueous based semi-permanent lowlighting dye composition containing at least one particulate in an amount sufficient to provide a visually distinctive appearance when applied to hair; and (b) an applicator for applying (a) to hair to provide gradations of deeper color approximating lowlights to the hair.

18. The kit of claim 17 wherein the semi-permanent dye composition is an aqueous based composition comprising at least one water soluble or dispersible dye operable to color hair when in contact therewith, and at least one particulate in an amount sufficient to provide a visually distinctive appearance when the semi-permanent dye composition is applied to the hair.

19. The kit of claim 18 wherein the water soluble or water dispersible dye is an HC dye, a basic dye, a direct dye, or mixtures thereof.

20. The kit of claim 18 wherein the particulate is an inorganic pigment, an organic pigment, or a filler.

21. The kit of claim 20 wherein the inorganic pigment is a Lake.

22. The kit of claim 20 wherein the inorganic pigment is an iron oxide.

23. A method for applying lowlights to hair comprising the steps of:
(a) combining an aqueous oxidizing agent composition and a lowlight dye composition wherein one or both of such compositions comprise a particulate in an amount sufficient to provide a mixture that has a visually distinctive appearance when applied to hair,
(b) applying the mixture of (a) to select strands of hair using an applicator having a handle and a face having a plurality of tines that are perpendicular to the head,
(c) leaving the mixture on the hair for a period of time ranging from 1 to 60 minutes; and
(d) rinsing the mixture from the hair.

24. The method of claim 23 wherein after the mixture is rinsed from the hair, residual oxidizing agent remains on the hair.

25. The method of claim 24 wherein the hair is treated with a shampoo composition containing at least one oxidative dye, but being free of any oxidizing agents reactive with the oxidative dye to form color whereby the oxidative dye present in the shampoo composition reacts with the residual oxidizing agent on the hair to form additional color.

26. The method of claim 25 wherein the shampoo composition comprises, by weight of the total composition, from about 0.01-20% cleansing surfactant, from about 0.001-20% of one or more oxidative dyes, from about 0.001-99% water.

27. The method of claim 23 wherein the aqueous oxidizing agent composition comprises an aqueous solution of hydrogen peroxide.

28. The method of claim 27 wherein the aqueous oxidizing agent composition comprises an aqueous emulsion of hydrogen peroxide.

29. The method of claim 23 wherein the particulate is an organic pigment, and inorganic pigment, or mixtures thereof.

30. The method of claim 29 wherein the organic pigment is a water insoluble FD&C or D&C color.

31. The method of claim 29 wherein the inorganic pigment is titanium dioxide or iron oxides.

32. The method of claim 30 wherein the pigment comprises a Lake.

33. The method of claim 30 wherein the pigment comprises ultramarine blue.

34. A method for lowlighting hair by applying to select strands of hair an aqueous based semi-permanent dye composition containing at least one particulate in an amount sufficient to provide a visually distinctive appearance when applied to the hair.

35. The method of claim 34 wherein the particulate is an organic pigment, inorganic pigment, or mixtures thereof.

36. The method of claim 34 wherein the aqueous based semi-permanent dye composition containing the particulate is bright blue, pink, lime green, or red.

37. A method for applying lowlights to hair comprising treating the hair with a lowlight dye mixture containing particulates in an amount sufficient to provide a composition having a visually distinctive appearance when applied to the hair.

38. The method of claim 37 wherein the lowlight dye mixture comprises an aqueous based composition comprising hydrogen peroxide, at least one primary intermediate and coupler for the formation of oxidative dye, and at least one water insoluble particulate for providing the visually distinctive appearance.

39. The method of claim 37 wherein the lowlight dye mixture comprises an aqueous based semi-permanent dye composition.

40. The method of claim 37 wherein the lowlight dye mixture comprises a mixture of an aqueous based oxidizing agent composition, an aqueous based oxidative dye composition, and a particulate persulfate composition.

41. The method of claim 38 wherein the water insoluble particulate is an inorganic pigment, an organic pigment, a particulate filler, or mixtures thereof.

42. The method of claim 40 wherein the particulate is present ranging from about 0.001-30% by weight of the total mixture.

43. An aqueous based lowlight dye composition comprising at least one dye operable to color hair and at least one pigment in an amount sufficient to provide a composition that is red, pink, green, yellow, purple, or blue.

43. The composition of claim 43 wherein the pigment is an organic Lake or iron oxide and the composition is pink.

44. The composition of claim 43 wherein the pigment is an organic Lake and the composition is blue.

45. The composition of claim 43 wherein the pigment is an iron oxide or organic Lake and the composition is green.
